(19) United States
(12) Patent Application Publication
(10) Pub. No.: US 2006/0064104 A1
(43) Pub. Date: Mar. 23, 2006

(54) Translating surgical mount

(76) Inventors: Richard J. Kana, Lexington, TX (US); Joseph W. Pieczynski II, Austin, TX (US)

Correspondence Address: ZIMMER TECHNOLOGY - REEVES, P. O. BOX 1268, ALEDO, TX, 76008, US

(21) Appl. No.: 10/937,057

(22) Filed: Sep. 9, 2004

(51) Int. Cl. A61F 5/00

(52) U.S. Cl. 606/86

(57) ABSTRACT

A translating surgical mount used to selectively position a surgical component in one of a plurality of positions adjacent to a surgical site.

### FIELD OF THE INVENTION

The invention relates to an apparatus and method for mounting surgical components. More particularly, the invention relates to a translating surgical mount used to selectively position a surgical component in one of a plurality of positions adjacent to a surgical site.

### BACKGROUND

A variety of surgical components may be used during a surgical procedure. Surgical components may include trial implants, drill guides, cutting guides, implants, and/or other surgical components. For example, surgical instruments are used in a variety of ways to accomplish a surgical procedure. For example, surgical instruments may be used to gauge the size of a surgical site, prepare a surgical site to receive an implant, and/or otherwise to accomplish the procedure. Likewise, a surgical implant for a particular location in a patient's body may be provided in a variety of sizes and shapes to fit a variety of patients. In the course of planning and providing surgical care, a surgeon must choose the appropriate size and shape of implant for a particular patient and prepare the surgical site to receive the implant. A set of instruments including individual instruments that each approximates a particular size and shape of implant may be provided to the surgeon to be used as a guide in determining the best implant for the patient. These same instruments, or another set, may include cutting, drilling, positioning, and/or other guidance to aid preparation of the surgical site for a particular one of the implants. For example, orthopaedic implants may include stems for anchoring the implant to a bone. Such stems often extend longitudinally into a long bone along the intramedullary canal. A set of trial instruments for such an implant may include stemmed trials having different shapes, sizes, and stem offsets. A set of preparation instruments for such an implant may include guides for guiding reaming the intramedullary canal, broaching a proximal cavity, drilling fixation holes, cutting box angles, and/or other preparation steps.

### SUMMARY

The present invention provides a translating surgical mount used to selectively position a surgical component in one of a plurality of positions adjacent to a surgical site.

In one aspect of the invention, a combination includes a fixed reference positionable adjacent a surgical site, a surgical component, and a translating surgical mount. The translating surgical mount includes means for engaging the fixed reference, means for engaging the surgical component, and means for locking the means for engaging the surgical component and the means for engaging the fixed reference in one of a plurality of translated relative positions.

In another aspect of the invention, a translating surgical mount for connecting a tibial drill guide to an intramedullary rod includes a base engageable with the tibial drill guide and a slide engageable with the intramedullary rod. The slide is mounted to the base for translation between a plurality of relative translated positions so that the base and slide may be relatively translated to position the tibial drill guide at a desired translated position relative to the intramedullary rod.

In another aspect of the invention, a combination includes a set of implants and an instrument. Each implant in the set includes an implant body and a stem. Each implant has a medial side and a lateral side. The set of implants includes implants with different medial/lateral positions of the stem relative to the body. The instrument includes a guide, a rod, and a translating surgical mount connecting the guide to the rod. The translating surgical mount includes means for translating the guide and rod relative to one another between a plurality of positions corresponding to the different medial/lateral positions of the stem relative to the body.

In another aspect of the invention, a method includes: providing a surgical component to be positioned at a surgical site; providing a fixed reference at the surgical site; providing a translating surgical mount having a base and a slide, the slide being mounted to the base for translation between a plurality of relative translated positions, the translating surgical mount further including a locking mechanism for releasably locking the slide and base in each of the plurality of relative translated positions; connecting the surgical component to the fixed reference with the translating surgical mount; translating the base relative to the slide to position the surgical component at a desired translated position relative to the fixed reference; and locking the position.

### BRIEF DESCRIPTION OF THE DRAWINGS

Various examples of the present invention will be discussed with reference to the appended drawings. These drawings depict only illustrative examples of the invention and are not to be considered limiting of its scope.

FIG. 1 is an exploded perspective view of an exemplary translating surgical mount according to the present invention;

FIG. 2 is a top plan view of the base of the exemplary translating surgical mount of FIG. 1;

FIG. 3 is a cross-sectional view of the exemplary translating surgical mount of FIG. 1;

FIG. 4 is an exploded perspective view of the exemplary translating surgical mount of FIG. 1 being assembled to an exemplary surgical instrument and intramedullary rod;

FIG. 5 is a perspective view of the exemplary translating surgical mount and instrument assembly of FIG. 4 placed at a surgical site; and

FIG. 6 is a perspective view of knee prosthesis.

### DESCRIPTION OF THE ILLUSTRATIVE EXAMPLES

Embodiments of a translating surgical mount provide means for adjustably mounting a surgical component adjacent to a surgical site. The translating surgical mount may be used at a variety of surgical sites such as at a hip joint, knee joint, vertebral joint, shoulder joint, elbow joint, ankle joint, digital joint of the hand or foot, fracture site, tumor site, and/or other suitable surgical site. The surgical component with which the translating surgical mount is used may include trial implants, drill guides, cutting guides, implants, and/or other surgical components.

The translating surgical mount may be used to position a surgical component relative to a fixed reference at the surgical site. For example, the translating surgical mount may be used to selectively position the surgical component at predetermined known locations relative to a clamp, pin, screw, hole, and/or other reference. For example, the translating surgical mount may be used to position the surgical component relative to a rod located in the intramedullary canal of a long bone.

The translating surgical mount may include means for selectively translating the surgical component between predetermined positions. For example, the mount may connect the surgical component to a fixed reference and include means for translating the surgical component relative to the reference. For example, the translating surgical mount may include first and second parts mounted together for relative translation. One of the parts may engage the reference and the other part may engage the surgical component. Translating the first part relative to the second part translates the surgical component relative to the reference. Translation of the first part relative to the second part may be controlled by one or more detents, screws, rack and pinion arrangements, dovetail slides, rolling bearings, and/or other suitable arrangement. For example, a multiple position detent may be provided to permit releasable locking of the first and second parts in multiple translated positions.

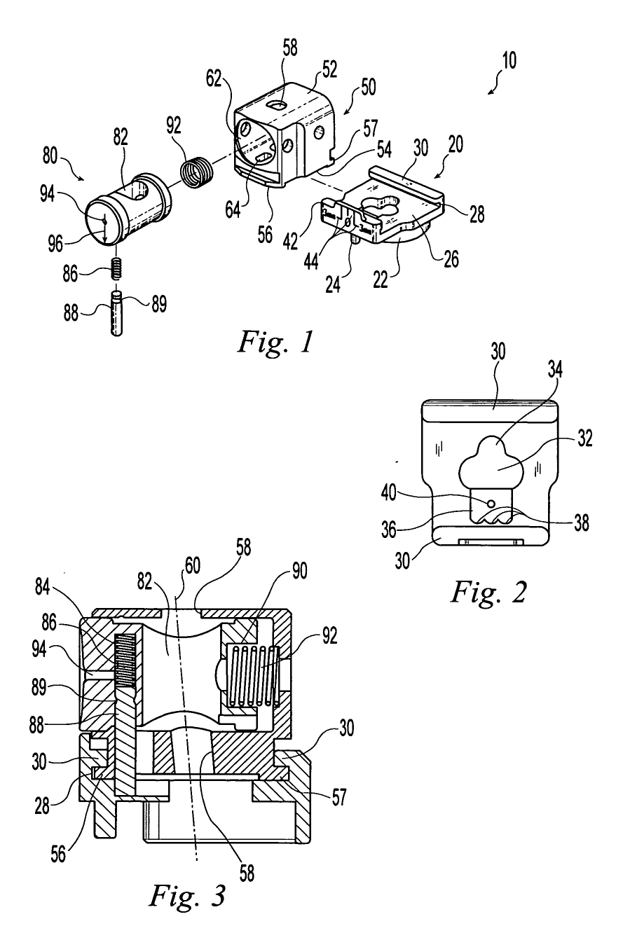

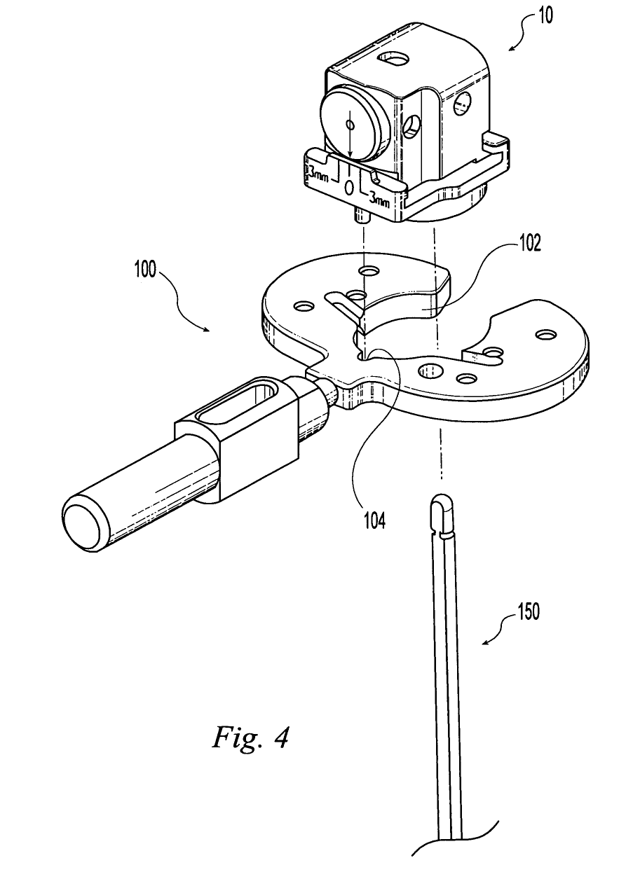

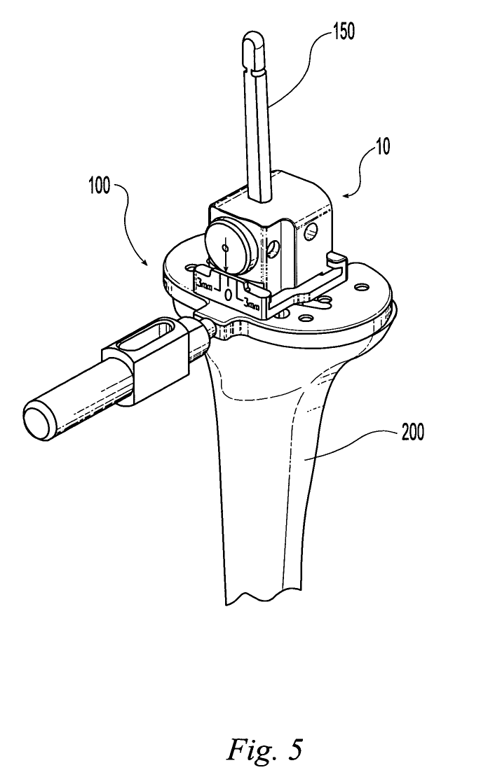

FIGS. 1-5 depict an exemplary translating surgical mount 10 configured to adjustably mount a tibial sizing/drill guide 100 adjacent to a knee joint. The exemplary translating surgical mount 10 is configured to position the tibial guide 100 relative to an intramedullary rod 150 inserted into the intramedullary canal of a tibia 200 (FIG. 5).

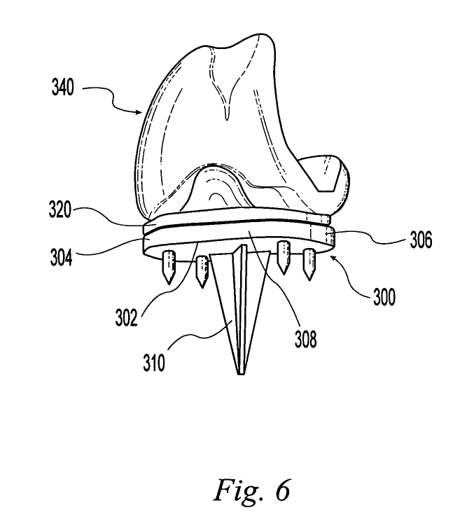

FIG. 6 depicts a knee prosthesis for use in total knee replacement surgery. Total knee replacement surgery typically involves replacing the proximal portion of the tibia 200 with a tibial plate implant 300, replacing the distal portion of the femur with a femoral implant 340, and interposing a bearing 320 between the tibial plate implant 300 and femoral implant 340. The tibial plate implant 300 includes a plate portion 302 having medial 304, lateral 306, anterior 308, and posterior (not shown) sides. A stem 310 may be provided to help anchor the tibial plate implant 300 on the tibia 200. The tibial plate implant 300 may be provided in a variety of sizes with different medial/lateral and anterior/posterior dimensions to optimize coverage of different sizes of tibias. In addition, the implants may be provided with the stem 310 attached at different locations to further optimize coverage of the tibia 200 since the intramedullary canal of the tibia 200 may not be located precisely under the middle of the plate portion 302 when it is positioned for best coverage. Any number of tibial plate implants 300 may be provided with the stems offset in any suitable direction. For example, tibial plate implants 300 may be provided with three stem 310 positions including centered, offset medially, and offset laterally.

The exemplary tibial guide 100 is configured to determine the appropriate size and medial/lateral positioning for a tibial plate implant 300 implant relative to the intramedullary canal and the outside periphery of the tibia 200. The exemplary translating surgical mount 10 is configured to position the tibial guide 100 at one of three user selectable positions corresponding to the centered, offset medially, and offset laterally stem positions. The exemplary translating surgical mount 10 reduces the number of tibial guides 100 required by allowing a single guide 100 to be used to test the fit for a plurality of different tibial plate implant 300 geometries. Without the use of the translating surgical mount 10, a guide 100 would be needed for each different tibial plate implant 300 geometry that is provided.

The translating surgical mount 10 includes a base 20, a slide 50 mounted to the base, and a pushbutton 80 mounted in the slide 50. The base 20, includes a semi-circular protrusion 22 projecting from the bottom of the base 20. An anti-rotation pin 24 projects from the bottom of the base 20 adjacent to the semi-circular protrusion 22. The semi-circular protrusion 22 and the anti-rotation pin 24 engage the tibial guide 100 in use. The base includes a “T”-slot 26 formed in its top surface for engaging the slide 50. The “T”-slot is defined by undercut slots 28 and overhanging rails 30. An elongated clearance hole 32 extends through the base 20 from top to bottom and is elongated medial/laterally. An optional tooling hole 34 may be provided to facilitate manufacturing. A recessed area 36 is formed into the surface of the base 20 inside the “T”-slot 26. The anterior side of the recessed area 36 is scalloped to form the notches 38 of a detent mechanism. A disassembly hole 40 is formed through the recessed area 36 to aid in disassembling the translating surgical mount 10. The front surface 42 of the base 20 includes indicia 44 to indicate the relative position of the base 20 and slide 50 in use.

The slide 50 includes a top surface 52 and a bottom surface 54. Anterior and posterior tongues 56, 57 are formed adjacent to the bottom surface 54. The tongues 56, 57 engage the undercuts 28 of the “T”-slot in the base 20 to permit the slide 50 to translate on the base 20. A “D”-shaped hole 58 extends through the slide 50 from the top surface 52 to the bottom surface 54 along an axis 60. The axis 60 of the “D”-shaped hole 58 is angled five degrees anteriorly in the anterior-posterior plane to permit the tibial guide 100 to sit flat on a proximal tibial bone cut with five degrees of posterior slope. The slide 50 also includes a posteriorly directed blind hole 62 for receiving the pushbutton 80 transverse to the axis 60. An anterior/posterior elongated hole 64 extends from the blind hole 62 to the bottom surface 54 of the slide to align with the notches 38 and recessed area 36 of the base 20 when the slide is mounted to the base.

The pushbutton 80 is a cylindrical member having an anterior/posterior elongated transverse through hole 82. The pushbutton 80 includes a blind index pin hole 84 for receiving an index spring 86 and an index pin 88 parallel to the through hole 82. the index pin 88 includes an annular groove 89 that may be used to aid assembly and disassembly of the translating surgical mount 10. The pushbutton 80 includes a blind mainspring hole 90 for receiving a mainspring 92. The pushbutton 80 also includes an assembly/disassembly hole 94 communicating with the index pin hole 84. The pushbutton 80 further includes an index mark 96.

The exemplary translating surgical mount 10 is assembled by placing the mainspring 92 into the slide 50 through the blind hole 62 followed by the pushbutton 80. The mainspring hole 90 of the pushbutton 80 will receive a portion of the mainspring 92. The pushbutton 80 is then depressed until the index pin hole 84 aligns with the anterior/posterior elongated hole 64 in the slide. The index spring 86 and index pin 88 may then be inserted through the bottom 54 of the slide 50, through the elongated hole 64, and into the index pin hole 84. With the index pin 88 in the index pin hole 84, the pushbutton 80 is captured in the slide 50. The slide 50 may then be slid partway onto the base 20 by engaging the slide tongues 56, 57 with the “T”-slot 26 of the base 20. The index pin 88 will abut the edge of the base 20 and prevent final assembly. By pressing the index pin 88 further into the index pin hole 84 against spring 86 tension, the index pin 88 will clear the edge of the base 20 and the slide 50 may be fully assembled onto the base 20. The index pin 88 is biased into the recessed area 36 by the spring 86 so that it engages one of the notches 38 and locks the relative position of the base 20 and slide 50. Optionally, a pin may be temporarily inserted through the assembly/disassembly hole 94 to engage the annular groove 89 of the index pin 88 to hold it in the index pin hole 84 to clear the edge of the base 20.

The exemplary translating surgical mount 10 is disassembled by pressing the pushbutton 80 and aligning the index pin with the disassembly hole 40 in the base 20. A pin may then be pressed through the disassembly hole 40 to press the index pin 88 into the index pin hole 84 against the spring 86. A temporary holding pin may be inserted through the assembly/disassembly hole 94 to engage the annular groove 89 of the index pin 88 and hold it in the index pin hole 84 to clear the edge of the recessed area 36. The slide 50 may then be slid off of the base 20. The temporary holding pin may be removed from the assembly/disassembly hole 94 to release the index pin 88 and spring 86. The index pin 88 and spring 86 may be removed from the pushbutton 80 through the bottom of the slide 50 and the pushbutton 80 and mainspring 92 may be withdrawn from the slide 50.

Assembly of the translating surgical mount 10 is easily accomplished without the use of separate fasteners, soldering, brazing, welding, or other separate fastening means. Each component of the mount 10 may be completely fabricated, heat treated, polished, passivated, and otherwise finished prior to assembly. Discoloration, warping, and other undesirable changes to the assembly associated with heating of the assembly are thus avoided. Likewise, accumulation of debris in the assembly from abrasive grit blasting, grinding, or otherwise doing finish work on the assembly are also avoided. The self-locking assembly configuration thus results in a clean, smoothly operating mechanism.

In use, the base 20 is engaged with a surgical component and the slide 50 is engaged with a fixed reference. The base 20 may be adjusted relative to the slide 50 and fixed reference by disengaging the index pin 88 from one of the notches 38 in the base 20, changing the base 20 and slide 50 relative positions, and reengaging the index pin 88 with one of the notches 38. In the exemplary arrangement, the intramedullary rod 150 is inserted along the intramedullary canal of the tibia 200 to establish a fixed reference. The base 20 is engaged with the tibial guide 100 by inserting the semi-circular protrusion 22 into a central hole 102 in the guide 100 and positioning the anti-rotation pin 24 in a corresponding notch 104 in the guide 100. The base 20 and guide 100 will now move together. This assembly is placed on the tibia 200 over the intramedullary rod 150. The slide 50 is engaged with the intramedullary rod 150 by inserting the rod through the elongated clearance hole 32 in the base and through the “D”-shaped hole 58 in the slide 50. The intramedullary rod 150 has a “D”-shaped cross section corresponding to the “D” shaped hole 58 to prevent rotation of the rod 150 in the hole 58. The tibial guide 100 visually demonstrates the coverage of a corresponding tibial implant on the tibial plateau. If there is overhang on one side, the surgeon may depress the pushbutton 80 and move the base 20 and tibial guide 100 relative to the slide 50 and intramedullary rod 150 in the desired direction to improve the tibial plateau coverage. When the pushbutton 80 is depressed, it drives the index pin 88 posteriorly to disengage the notches 38 in the base 20. The anterior/posterior elongated hole 64 in the slide 50 allows the index pin 88 to move posteriorly. The base 20 and tibial guide 100 may be shifted medially or laterally to a desired position. The index mark 96 on the pushbutton 80 aligns with the indicia 44 on the base to indicate the relative positions of the base 20 and slide 50. The medially/laterally elongated clearance hole 32 in the base provides clearance for the intramedullary rod 150 to permit the base 20 and guide 100 to be shifted. When the pushbutton 80 is released, the index pin 88 is biased into engagement with the notches 38 to again lock the relative positions of the base 20 and slide 50. If the surgeon desires to change the size of the tibial drill guide 100, the translating surgical mount 10 and the tibial drill guide 100 may be slid off of the intramedullary rod 150. The translating surgical mount 10 may be mounted to a different size drill guide 100 and the assembly replaced on the intramedullary rod 150. The new size may then be best positioned using the translating surgical mount 10.

Although examples of a translating surgical mount and its use have been described and illustrated in detail, it is to be understood that the same is intended by way of illustration and example only and is not to be taken by way of limitation. The invention has been illustrated in use to position a tibial guide medially and laterally relative to an intramedullary rod inserted into the intramedullary canal of a tibia at a knee joint. However, the translating surgical mount be configured to position a tibial guide anteriorly and posteriorly or in any other suitable direction or position. Likewise, the translating surgical mount may be configured for use at other locations within a patient's body to position other surgical components. Accordingly, variations in and modifications to the translating surgical mount and its use will be apparent to those of ordinary skill in the art, and the following claims are intended to cover all such modifications and equivalents.

What is claimed is:

1. In combination:
a fixed reference positionable adjacent a surgical site;
a surgical component; and
a translating surgical mount for positioning the surgical component relative to the fixed reference at the surgical site, the translating surgical mount comprising:
means for engaging the fixed reference;
means for engaging the surgical component, the means for engaging the surgical component being mounted for translation relative to the means for engaging the fixed reference, the means for engaging the surgical component being translatable relative to the means for engaging the fixed reference between a plurality of translated relative positions; and
means for locking the means for engaging the surgical component and the means for engaging the fixed reference in one of the plurality of translated relative positions.

2. The combination of claim 1 wherein the fixed reference comprises an intramedullary rod and the surgical component comprises a tibial drill guide.

3. The combination of claim 2 wherein the means for engaging the fixed reference and the means for engaging the surgical component are relatively translatable medial/laterally between a plurality of medial/lateral offset positions to position the intramedullary rod and tibial drill guide in one of the plurality of medial/lateral offset positions.

4. A translating surgical mount for connecting a tibial drill guide to an intramedullary rod, the intramedullary rod being inserted into a bone adjacent to a surgical site, the translating surgical mount comprising:
a base engageable with the tibial drill guide; and
a slide engageable with the intramedullary rod, the slide being mounted to the base for translation between a plurality of relative translated positions so that the base and slide may be relatively translated to position the tibial drill guide at a desired translated position relative to the intramedullary rod.

5. The translating surgical mount of claim 4 wherein the slide and base are mounted for relative medial/lateral translation between a plurality of medial/lateral offset positions to position the tibial drill guide in a plurality of medial/lateral offset positions relative to the intramedullary rod.

6. The translating surgical mount of claim 5 wherein the base and slide further comprise a detent mechanism able to releasably lock the base and slide in each of the plurality of medial/lateral offset positions.

7. The translating surgical mount of claim 6 further comprising:
a plurality of notches formed in the base;
an index pin releasably engageable with each notch in the plurality of notches formed in the base, the index pin being movable between a position in which the index pin engages the notches and a position in which the index pin is disengaged from the notches; and
a spring biasing the index pin into engagement with the notches.

8. The translating surgical mount of claim 7 wherein the base further comprises a “T”-slot and the slide further comprises a tongue engaging the “T”-slot to guide the slide and base in linear medial lateral relative translation.

9. The translating surgical mount of claim 7 wherein the index pin is mounted to a pushbutton, the pushbutton being mounted to the slide.

10. The translating surgical mount of claim 7 wherein the slide includes a bore and dovetail rails, the base includes a dovetail slot engaging the dovetail rails, and the base, slide, index pin, and pushbutton form an self-locking assembly in which the index pin retains the pushbutton within the bore in the slide and the index pin retains the slide dovetail rails in engagement with the base dovetail slot.

11. In combination:
a set of implants, each implant in the set including an implant body and a stem, each implant having a medial side and a lateral side, the set of implants including implants with different medial/lateral positions of the stem relative to the body;
an instrument including a guide, a rod, and a translating surgical mount connecting the guide to the rod, the translating surgical mount including means for translating the guide and rod relative to one another between a plurality of positions corresponding to the different medial/lateral positions of the stem relative to the body.

12. The combination of claim 11 wherein the set of implants includes implants with different implant body sizes and the instrument includes a plurality of guides corresponding to the different implant body sizes, each of the plurality of guides being interchangeably connectable to the translating surgical mount.

13. A method comprising:
providing a surgical component to be positioned at a surgical site;
providing a fixed reference at the surgical site;
providing a translating surgical mount having a base and a slide, the slide being mounted to the base for translation between a plurality of relative translated positions, the translating surgical mount further including a locking mechanism for releasably locking the slide and base in each of the plurality of relative translated positions;
connecting the surgical component to the fixed reference with the translating surgical mount;
translating the base relative to the slide to position the surgical component at a desired translated position relative to the fixed reference; and
locking the position.

14. The method of claim 13 wherein the fixed reference comprises an intramedullary rod inserted in the intramedullary canal of a tibia and the surgical component comprises a tibial guide having a periphery corresponding to a periphery of a tibial implant and the method further comprises:
visualizing the coverage of the tibial guide on the tibia;
translating the base and slide to obtain a desired coverage of the tibia;
determining the amount of medial/lateral offset there is between the tibial guide and intramedullary rod; and
selecting a tibial implant having a corresponding offset.

15. The method of claim 14 wherein the translating surgical mount further comprises a plurality of notches formed in the base and an index pin mounted to the slide, the index pin being movable between a position in which the index pin engages the notches and a position in which the index pin is disengaged from the notches; and a spring biasing the index pin into the position in which the index pin engages the notches, the method further comprising:
pressing the pushbutton to disengage the index pin from the notches;
translating the base relative to the slide medial/laterally to obtain a desired coverage of the tibia; and
releasing the pushbutton to lock the medial/lateral position of the base and slide.

16. The method of claim 14 further comprising:
removing the tibial guide;
substituting a tibial guide of a different size;
translating the base relative to the slide medial/laterally to obtain a desired coverage of the tibia.

17. A translating surgical mount for positioning a surgical component relative to a fixed reference at a surgical site, comprising:
means for engaging the fixed reference;
means for engaging the surgical component, the means for engaging the surgical component being mounted for translation relative to the means for engaging the fixed reference, the means for engaging the surgical component being translatable relative to the means for engaging the fixed reference between a plurality of translated relative positions; and
means for locking the means for engaging the surgical component and the means for engaging the fixed reference in one of the plurality of translated relative positions.
